(19) United States
(12) Patent Application Publication
(10) Pub. No.: US 2024/0410890 A1
(43) Pub. Date: Dec. 12, 2024

(54) SCREENING SYSTEMS AND METHODS FOR HPV-ASSOCIATED CERVICAL DISEASE

(71) Applicant: OncoGenesis, Inc., Salt Lake City, UT (US)

(72) Inventors: Paul VICHI, Lincoln, VT (US); Peter P. GOMBRICH, Salinas, CA (US)

(21) Appl. No.: 18/818,142

(22) Filed: Aug. 28, 2024

Related U.S. Application Data
(63) Continuation of application No. 17/385,348, filed on Jul. 26, 2021, now abandoned.
(60) Provisional application No. 63/149,479, filed on Feb. 15, 2021.
(60) Provisional application No. 63/056,983, filed on Jul. 27, 2020.

(51) Int. Cl. G01N 33/574; A61B 10/00; A61B 10/02; B01L 3/00; B01L 3/02; C12Q 1/6806; C12Q 1/70; G01N 1/00; G01N 33/569

(52) U.S. Cl. CPC G01N 33/57411; A61B 10/0045; A61B 10/0291; B01L 3/022; B01L 3/5027; C12Q 1/6806; C12Q 1/708; G01N 33/56983; B01L 2200/0647; B01L 2200/0689; B01L 2300/0681; C12Q 2565/629; G01N 2001/002; G01N 2333/025

(57) ABSTRACT

Devices and methods described herein provide improved methods of screening for cervical disease. In certain embodiments, a sample transfer and preparation vial is provided, enabling self-collection and pre-processing of cervical samples to expedite sample processing and eliminate the need for a patient to travel to a medical facility for screening. In certain embodiments, an analysis cartridge having a multiplexed biomarker panel and an immunoassay-based analyzer are provided for sample analysis. The multiplexed biomarker panel provides high sensitivity and specificity to enable effective screening with a single procedure and thus, eliminates the need for multiple tests. In certain embodiments, a method of screening for cervical disease is provided, utilizing the aforementioned multiplexed biomarker panel. The method includes detecting levels of at least two biomarkers in a cervical sample, wherein one of the biomarkers is an oncoprotein from a high-risk strain of human papilloma virus (HPV) and another is a cellular protein.

### CROSS-REFERENCE TO RELATED APPLICATIONS

This application is a continuation of U.S. patent application Ser. No. 17/385,348, filed Jul. 26, 2021, which claims benefit of U.S. Provisional Patent Application No. 63/149,479, filed Feb. 15, 2021, and U.S.

Provisional Patent Application No. 63/056,983, filed Jul. 27, 2020, each of which is herein incorporated by reference in its entirety.

### REFERENCE TO SEQUENCE LISTING

This application contains references to amino acid and nucleic acid sequences which have been submitted as the sequence listing text file entitled “Sequence Listing_1-14_XML”, file size 21.0 Kilobytes (KB), created Aug. 27, 2024, which is hereby incorporated by reference into this application in its entirety.

### BACKGROUND

### Field

Embodiments of the present disclosure generally relate to methods, devices, and systems for detection of cervical disease, and more particularly, screening of human papillomavirus (HPV) and HPV-associated cervical disease.

### Description of the Related Art

Cervical cancer is the fourth most frequent cancer in women and seventh overall with over 570,000 new cases and 300,000 associated deaths annually worldwide. Current preventive techniques for cervical cancer include repeated screening with conventional methods such as liquid-based cytology and Papanicolaou (Pap) smear-based cytology. However, as currently utilized, these methods fail to meet the demands of low-resource settings, which account for almost 90% of cervical cancer deaths in the world.

Cytological cervical cancer screening techniques, which have been available since the 1940's, involve the subjective identification of disease via microscopic observation of stained cervical cells for morphological evidence of abnormal cervical cells or precancerous cervical lesions (i.e., cervical disease). HPV screening techniques, on the other hand, may provide an objective component to cervical screening, but currently lack specificity for cervical disease due to the high worldwide incidence of HPV infections, the majority of which are cleared without medical intervention or associated health problems. Thus, although virtually all cervical cancers will screen positive for HPV, the majority of infections will not result in cervical carcinoma. Hence, when used as a primary screening tool to predict cervical cancer, the lack of specificity of current HPV screening techniques requires subsequent testing and confirmation with cytological techniques or other metrics in order to accurately detect disease rather than simply infection.

In combination, cytology and HPV screening can be effective for cervical screening when administered on a regular basis. However, these methods are very costly, requiring trained healthcare or laboratory personnel and significant investment in infrastructure, which are not readily available in low-resource regions. In addition, due to the subjective nature of cytological techniques, false negative results lead to inaccuracies in disease identification, demanding further resources for resolution thereof.

Accordingly, what is needed in the art are improved systems, devices, and methods for screening of HPV-associated cervical disease.

### SUMMARY

The present disclosure generally relates to systems, devices, and methods for screening of HPV-associated cervical disease.

In one embodiment, a method of detecting cervical disease is provided. The method includes contacting a biological sample obtained from a patient with a plurality of binding agents specific to at least one human papillomavirus (HPV) oncoprotein from three or more high-risk HPV (hr-HPV) isoforms, at least one independent prognostic indicator for cervical disease, and at least one independent indicator of sample viability. The method further includes measuring specific binding between the plurality of binding agents and each of the HPV oncoprotein, the independent prognostic indicator for cervical disease, and the independent indicator of sample viability to determine levels of the HPV oncoprotein, independent prognostic indicator for cervical disease, and the indicator of sample viability in the biological sample.

In one embodiment, a point-of-care device for detecting cervical disease in a biological sample is provided. The point-of-care device includes a disposable cartridge having a chromatographic membrane formed of nitrocellulose, silica, paper, micro-patterned silicon, or polymeric materials. The chromatographic membrane is further conjugated with a plurality of binding agents specific to at least one human papillomavirus (HPV) oncoprotein from three or more high-risk HPV (hr-HPV) isoforms, at least one independent prognostic indicator for cervical disease, and at least one independent indicator of sample viability.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIGS. 1A-1B illustrate schematic cross-sectional views of exemplary cervical cell sample transfer and preparation devices, according to embodiments of the present disclosure.

FIG. 2 illustrates a schematic cross-sectional view of an exemplary analysis cartridge for analysis of a cervical cell sample, according to embodiments of the present disclosure.

FIG. 3 illustrates an exemplary binding arrangement of substrate surface molecules with a target biomarker, according to embodiments of the present disclosure.

FIGS. 4A-4B illustrate tables of exemplary individual or multiplexed target biomarkers for analysis of a cervical sample, according to embodiments of the present disclosure.

FIG. 5 illustrates a table of exemplary antibodies that may be utilized as capture and/or detection molecules for binding with target biomarkers, according to embodiments of the present disclosure.

FIG. 6 illustrates conserved regions of the HPV E7 gene. Conserved N-terminal regions CR1 and CR2 and C-terminal zinc binding domains (CXXC) are shown in black. (Roman A., Munger K. 2013, Virology 445; 138-168).

FIG. 7A illustrates a nucleic acid sequence of messenger RNA (mRNA) encoding HPV type 16 E7 isolate IR-32, Acc #: KM058635, SEQ ID NO 1, according to embodiments of the present disclosure.

FIG. 7B illustrates an amino acid sequence for HPV type 16 E7 protein, ID #: AIQ82815.1, SEQ ID NO 2, according to embodiments of the present disclosure.

FIG. 7C illustrates a nucleic acid sequence of mRNA encoding HPV type 18 E7 isolate B8890, Acc #KC662605.1, SEQ ID NO 3, according to embodiments of the present disclosure.

FIG. 7D illustrates an amino acid sequence for HPV type 18 E7 protein, ID #: AGM34461.1, SEQ ID NO 4, according to embodiments of the present disclosure.

FIG. 7E illustrates a nucleic acid sequence for HPV type 31 E7 gene isolate LNS8465006_HPV31, Acc #: KC662598, SEQ ID NO 5, according to embodiments of the present disclosure.

FIG. 7F illustrates an amino acid sequence for HPV type 31 E7 protein, ID #: AMG34454.1, SEQ ID NO 6, according to embodiments of the present disclosure.

FIG. 7G illustrates a nucleic acid sequence for HPV type 45 E7 gene isolate B3587, Acc #: KC662609, SEQ ID NO 7, according to embodiments of the present disclosure.

FIG. 7H illustrates an amino acid sequence for HPV type 45 E7 protein, ID #: AGM34465, SEQ ID NO 8, according to embodiments of the present disclosure.

FIG. 8A illustrates a nucleic acid sequence of mRNA for cytokeratin 17 (i.e., KRT17), Acc #: NM_000422, SEQ ID NO 9, according to embodiments of the present disclosure.

FIG. 8B illustrates an amino acid sequence for cytokeratin 17, SEQ ID NO 10, according to embodiments of the present disclosure.

FIG. 9A illustrates a nucleic acid sequence of mRNA for survivin (i.e., baculoviral IAP repeat containing 5 (BIRC5)), Acc #: NM_001168.3, SEQ ID NO 11, according to embodiments of the present disclosure.

FIG. 9B illustrates an amino acid sequence for survivin (BIRC5), SEQ ID NO 12, according to embodiments of the present disclosure.

FIG. 10A illustrates a nucleic acid sequence for HPV type 16 L1 capsid gene isolate (i.e., HPV16 L1), Acc #: NC_001526.4, SEQ ID NO 13, according to embodiments of the present disclosure.

FIG. 10B illustrates an amino acid sequence for HPV type 16 L1 capsid protein, ID #: NP_041332.2, SEQ ID NO 14, according to embodiments of the present disclosure.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Embodiments described herein provide systems and methods for improved cervical disease screening.

### Protein-Based Screening for HPV-Associated Cervical Disease

Conventional techniques for cervical cancer screening, including cytology and high-risk human papillomavirus (hr-HPV) nucleic acid (e.g., DNA or RNA) detection, require the performance of multiple screens and/or combination with additional tests. Particularly, cytology is subjective in approach to disease identification and frequently results in false positives and inaccuracies, and although HPV is an etiological agent for cervical cancer, nucleic acid detection thereof only determines the presence of HPV infection and is not necessarily indicative of cancer. Thus, these techniques require subsequent testing and confirmation, which compounds the difficulty in performing effective screening and increases the cost thereof. In addition, these techniques are labor and time intensive, requiring advanced infrastructure and trained medical or laboratory personnel to complete, which are not readily available in low-resource regions of the world. As a result, effective cervical cancer screening is generally inaccessible in these low-resource regions, where a large percentage of cervical cancer cases and an even greater percentage of cervical cancer deaths occur.

The use of protein biomarkers in cervical diagnostics can aid in the early detection of cervical lesions (i.e., disease) posing a risk of cervical cancer. Certain proteins can provide an objective diagnostic component with high sensitivity and specificity that can be utilized as a triage or even primary screening tool, reducing unnecessary tests and procedures for diagnostic confirmation. For example, cytokeratin protein 17 (K17) is upregulated in cervical cell transformation and is a novel cervical biomarker specific and sensitive for the detection of high-grade squamous intraepithelial lesions (HSIL) and squamous cell carcinomas. K17 functionally contributes to the degradation of p27 (kip1), a tumor cell cycle-related tumor suppressor, and also in polarizing the immune system. Accordingly, K17 may serve as a strong negative prognostic indicator for cervical cancer survival or recurrence independent of cancer stage.

The HPV lifecycle provides further opportunity to utilize specific viral proteins as independent markers to identify persistent HPV infections posing a risk of cervical cancer development. For example, oncoproteins E6 and E7, whose products are integral for viral replication, regulation, and host cell modification, bind to important cellular proteins p53^{16,17} and Rb¹⁸, leading to degradation thereof and disruption of several key cellular pathways including p53 and Rb/E2F related gene expression. HPV E6 and E7 oncoprotein-mediated interference of these pathways leads to chromosomal instability, loss of cell cycle, and loss of apoptotic and proliferative controls, which are hallmarks of cell transformation. Therefore, HPV oncoproteins E6 and E7 can be accurate indicators of persistent HPV infection that, when combined or multiplexed with other biomarkers such as cytokeratin K17, reflect HPV impact on specific cellular pathways and level of cervical disease that may otherwise require multiple tests to attain similar clinical information.

The devices and methods described herein provide improved methods of point-of-care (POC) screening for cervical disease utilizing a multiplexed biomarker approach. In certain embodiments, a cervical sample transfer and preparation device is provided, enabling collection of personal cervical samples from the patient's own home and eliminating the need for the patient to travel to a medical facility for sample collection, as well as the need for laboratory sample pre-processing. In certain embodiments, a sample cartridge having a multiplexed biomarker panel and an analyzer are provided for POC cervical sample analysis, facilitating both lab and non-lab-based sample analysis. The multiplexed biomarker panel described herein provides high sensitivity and specificity, enabling effective screening with a single procedure and thus, reduces or eliminates the need for multiple tests for confirmation.

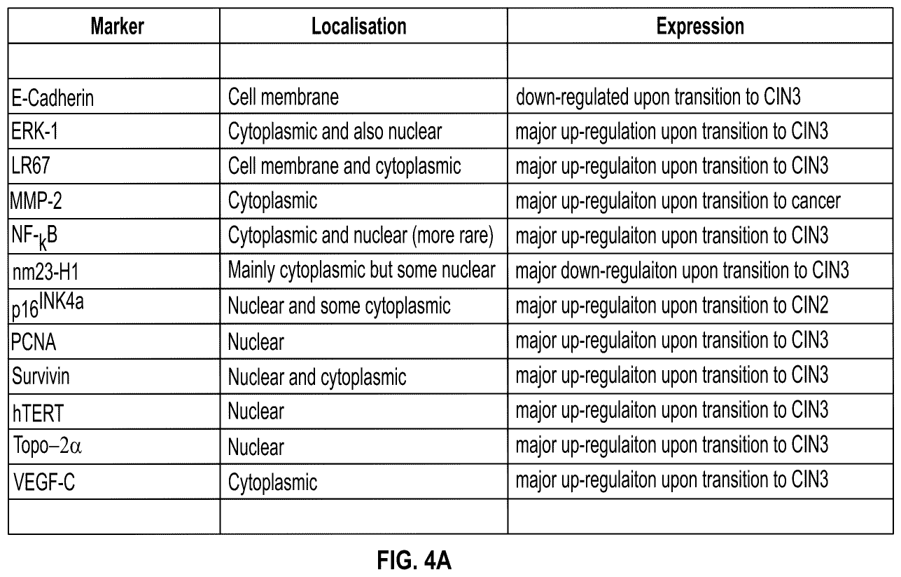

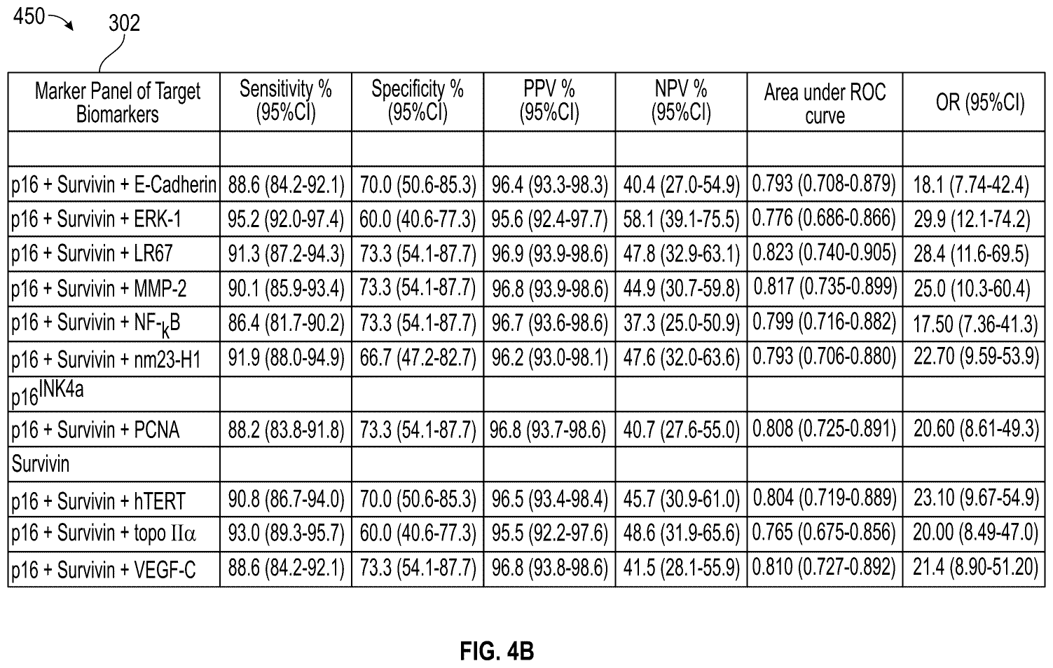

In certain embodiments, a method for screening of cervical disease is provided, utilizing the aforementioned multiplexed biomarker panel. The method includes detecting the level of at least two biomarkers in the cervical sample, wherein one of the biomarkers is an oncoprotein from a high-risk strain of human papilloma virus (HPV), such as E7, and another is a cellular protein, whose level is affected by the presence of HPV viral oncoproteins E7 or E6. Examples of cellular proteins of interest include pRb, p53, p16, cytokeratin 17, survivin, ki67, Tert, Erk, and LR67, (shown in FIGS. 4A and 4B) the levels of which are altered in cervical intraepithelial neoplasia (CIN) and cervical cancers or which themselves result in changes in the expression of other downstream cellular proteins. The method further provides quantification of the detected biomarkers, thus enabling better characterization of the disease status of the cervical sample to determine appropriate medical action. In certain embodiments, detection of target biomarkers is sufficiently characterized through qualitative means, such as by visual color development, staining, and the like.

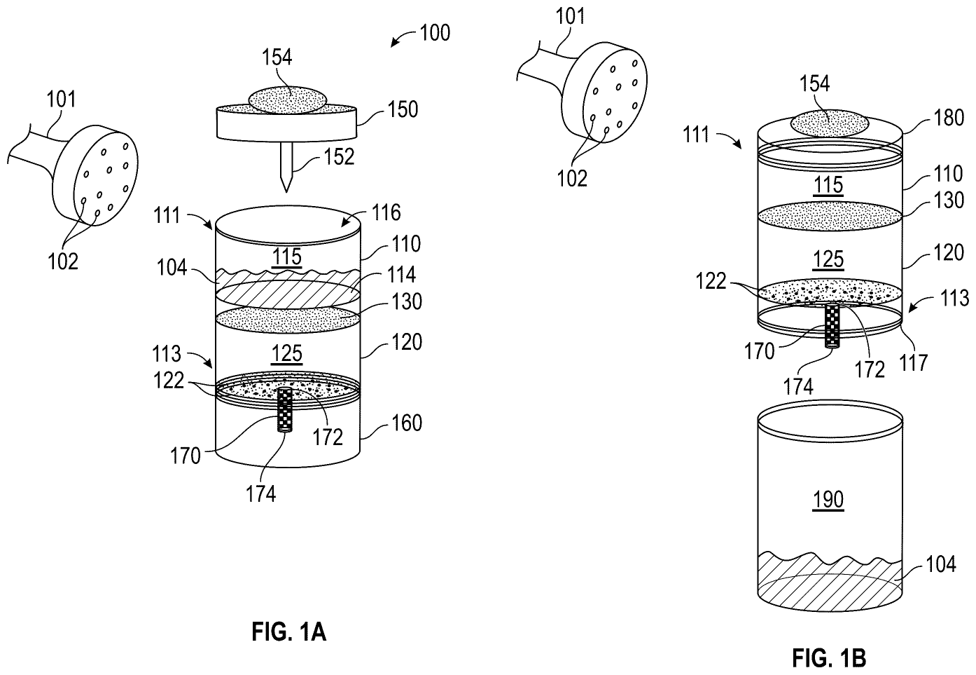

FIGS. 1A and 1B illustrate schematic cross-sectional diagrams of exemplary sample transfer and preparation devices 100 and 101, respectively, for transport and preparation (e.g., pre-processing) of a cervical sample 102, according to certain embodiments. The exemplary sample transfer and preparation devices 100 and 101 may be utilized in combination with other devices and methods of the present disclosure.

In certain embodiments, a cervical sample 102 is first collected by a patient or medical professional utilizing a personal cervical sample (e.g., cervical cell) collection device 103 and/or kit, and may be collected at the patient's home or other suitable location. Upon collection of the cervical sample 102 (e.g., cervical cells), the cervical sample 102 may be placed in a sample vial or in the sample transfer and preparation devices 100 or 101 for transfer to a POC testing center or laboratory for analysis, thus enabling access to cervical screening from the comfort of the patient's own home. A suitable cervical sample collection device and kit that can be used in combination with the present disclosure is described in U.S. application Ser. No. 15/456,259 to OncoGenesis Inc., of Morgan Hill, California, which is hereby incorporated by reference.

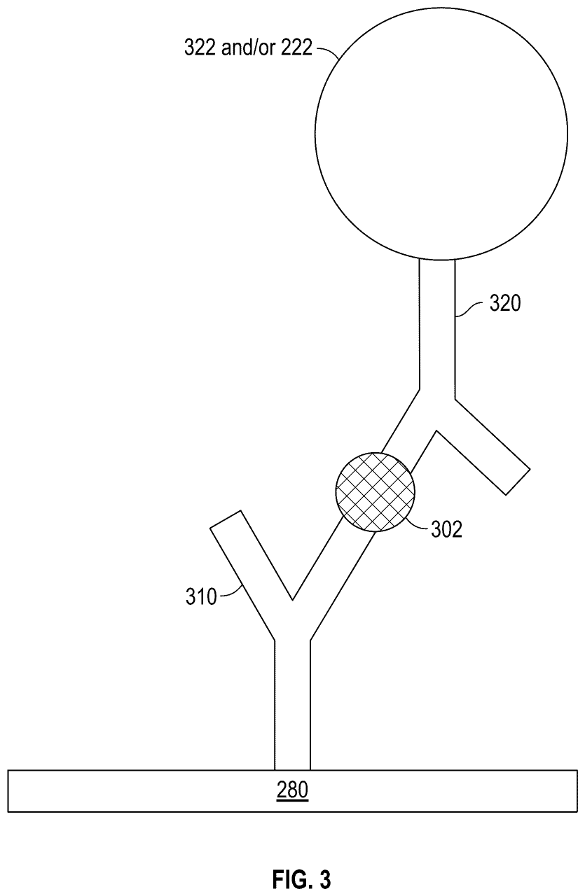

Cervical samples often contain significant levels of unwanted material such as cell fragments, red blood cells (RBCs), non-target cells, bacteria, as well as other biological debris that can complicate the detection of target biomarkers. Thus, collected cervical samples are usually pre-processed prior to analysis to remove these unwanted materials, which is usually performed in a laboratory setting. The exemplary sample transfer and preparation devices 100 and 101 described below, in combination with the exemplary analysis cartridge 300 depicted in FIG. 3, eliminate the need for such laboratory-based pre-processing by performing several functions to facilitate subsequent target biomarker recovery, detection, and quantification. For example, in certain embodiments, the sample transfer and preparation devices 100 and 101 and/or analysis cartridge 300 described herein are configured to filter large and unwanted materials, disrupt cellular clusters, remove red blood cells, and isolate desired target cells (e.g., squamous epithelial cells or endocervical cells) for enrichment thereof. Accordingly, the sample transfer and preparation devices 100 and 101 and analysis cartridge 300 described below enable efficient and on-the-fly preparation of cervical samples without the need for laboratory pre-processing operations.

In the exemplary embodiment illustrated in FIG. 1A, the transfer and preparation device 100 is a complete and standalone vial configured to have the collected cervical sample 102 loaded directly therein by a patient or clinician for transport to a POC testing center or laboratory. Accordingly, the cervical sample 102 may be transported in the transfer and preparation device 100 without the use of a separate sample vial or tube.

The transfer and preparation device 100 is generally a tubular container having a primary chamber 110 and a lysis chamber 120 separated (e.g., divided) by a filtering membrane 130. The primary chamber 110 is configured to seal and mix the loaded cervical sample 102 within a preservation solution 104 disposed in a volume 115 between the filtering membrane 130 and an upper end 111 of the transfer and preparation device 100. Examples of suitable preservative solutions 104 include methanol-, ethanol-, and/or isopropanol-based cytological preservatives. Prior to use, the preservative solution 104 may be packaged within the volume 115 between a breakable lower blocking membrane 114 and a removable upper blocking membrane 116. The lower blocking membrane 114 acts as a temporary block or seal to maintain the preservative solution 104 in the volume 115, providing an accessible reservoir for the user to rinse a cervical sample collection device 103 containing the cervical sample 102 after removal of the upper blocking membrane 116.

Upon loading of the cervical sample 102, the primary chamber 110, and thus the entire transfer and preparation device 100, is sealable at the upper end 111 by an upper cap 150. As shown in FIG. 1A, in certain embodiments, the upper cap 150 includes a rigid, needle-like appendage 152 on an underside thereof with sufficient length to rupture the lower blocking membrane 114 but not damage the filtering membrane 130. Accordingly, securing the upper cap 150 at the upper end 111 will cause the appendage 152 to rupture the lower blocking membrane 114, enabling the flow of cervical sample 102 from the volume 115 towards the filtering membrane 130 and into the lysis chamber 120. In certain embodiments, the upper cap 150 further includes a diaphragm (e.g., bulb) 154 configured to provide positive and/or negative pressure to the volume 115 upon mechanical actuation thereof, thus facilitating flow of the cervical sample 102 through the transfer and preparation device 100.

The filtering membrane 130 is configured to filter unwanted material from the cervical sample 102, such as mucous, debris, and other unwanted cellular matter as the cervical sample 102 passes therethrough. In certain embodiments, the filtering membrane is formed of polypropylene, polyimide, polyamide, nylon, or other suitable polymeric materials. The filtering membrane 130 generally has a pore size configured to enable flow of desired materials therethrough (e.g., cervical cells) while blocking entry of materials larger than the pore size. In certain embodiments, the filtering membrane 130 has a pore size of about 1600 μm, thus preventing material larger than 1600 μm from traversing between the primary chamber 110 and the lysis chamber 120.

The lysis chamber 120 is utilized to lyse and maintain (e.g., preserve) proteins (biomarkers) of the cervical sample 102 for transfer to an analysis cartridge, such as the analysis cartridge 300. In certain embodiments, a lyophilized lysis reagent 122 is deposited within a volume 125 of the lysis chamber 120, which may be solubilized by the cervical sample 102 flowed therein from the primary chamber 110. In certain other embodiments, the lysis reagent 122 is solubilized in solution contained within or added to the volume 125 by a user. The lysis reagent 122 is configured to disrupt target cell membranes within the cervical sample 102 to release cytoplasmic and membrane proteins, and in certain embodiments, nuclear proteins, free of degradation. Examples of suitable lysis reagents 122 include ionic (sodium dodecyl sulfate, deoxycholate, sarkosyl), nonionic (Triton X-100, DDM digitonin, Tween 20, Tween 80, NP-40), zwitterionic (CHAPS), or chaotropic (urea) detergents, buffers, protease inhibitors, and the like.

In certain embodiments, the lysis chamber 120 is further coupled to a removable lower cap 160 at a lower end 113 of the transfer and preparation device 100, which when removed exposes a threaded Luer-lock or Luer-slip type connection port 170 extending from the lysis chamber 120. The lower cap 160 protects the connection port 170 and provides stability to the transfer and preparation device 100 prior to transfer of the cervical sample 102 to an analysis cartridge. The connection port 170 is configured to connect the transfer and preparation device 100 with the analysis cartridge and enables efficient transfer of the cervical sample 102 therebetween.

In certain embodiments, the connection port 170 includes a porous frit 172 disposed at an upper end thereof where the connection port 170 is joined with the lysis chamber 120. The porous frit 172 is configured to filter the cervical sample 102 and any flow-through material prior to entering an analysis cartridge. Material filtered by the porous frit 172 may include cell nuclei, membrane, nucleic acids, or other cellular debris or precipitates. In certain embodiments, the connection port 170 further includes a blocking membrane 174 disposed at a lower end thereof opposite the upper end. The blocking membrane 174 may be formed of a similar material to blocking membranes 114 and 116, thus preventing fluid flow through the connection port 170 until the connection port 170 is attached to an analysis cartridge for subsequent analysis. For example, upon connection of the transfer and preparation device 100 with an analysis cartridge, the blocking membrane 174 may be punctured by a feature disposed in the analysis cartridge, such as a pin, to enable transfer of the cervical sample 102 therebetween.

In operation, the patient, upon collecting the cervical sample 102 with a personal cervical sample collection device 103, may transfer the cervical sample 102 from the cervical sample collection device 103 to the primary chamber 110 of the transfer and preparation device 100. In certain embodiments, the patient may simply swirl or rinse a tip of the cervical sample collection device 103 containing the cervical sample 102 in the preservation fluid 104 after removing the upper blocking membrane 116. In certain embodiments, the patient may utilize a disposable transfer pipette or other suitable device to transfer the collected cervical sample 102 to the preservation fluid 104.

Upon loading of the cervical sample 102, the transfer and preparation device 100 is sealed with the upper cap 150, causing the lower blocking membrane 114 to rupture. Rupture of the lower blocking membrane 114 facilitates the flow of the mixed cervical sample 102 through the filtering membrane 130 and into the lysis chamber 120 by gravity and/or other applied force. For example, in certain embodiments, the patient may repeatedly press the diaphragm 154 after sealing the transfer and preparation device 100 with the upper cap 150, thus creating a positive pressure to push the mixed cervical sample 102 into the lysis chamber 120 (e.g., through the filtering membrane 130). In the process, unwanted material larger than a pore size of the filtering membrane 130, for example, 1600 μm, is filtered by the filtering membrane 130, and prevented from traversing into the volume 125 of the lysis chamber 120. Target cells in solution smaller than 1600 μm, however, are able to flow through the filtering membrane 130 and are collected on the floor of the lysis chamber 120.

In certain embodiments, after reaching the lysis chamber 120, the mixed and filtered cervical sample 102 solubilizes dried lysis reagents 122 pre-deposited in the lysis chamber 120 to facilitate cell lysis of target cells. In certain other embodiments, the cervical sample 102 mixes with lysis reagents 122 contained in the lysis chamber 120 in solution. Accordingly, the cervical sample 102 may filtered and lysed in the transfer and preparation device 100 prior to and/or during transport to a POC testing center, wherein the cervical sample 102 is subsequently analyzed after transferring the lysate to a disposable microfluidic analysis cartridge (e.g., analysis cartridge 300).

FIG. 1B illustrates another exemplary transfer and preparation device 101 which can be utilized in combination with other devices and methods of the present disclosure, according to certain embodiments. The transfer and preparation device 101 is substantially similar to the transfer and preparation device 100, but is configured to function as a cap (e.g., attachment) for a sample transfer vial 190 rather than act as a standalone vial. Accordingly, the lower end 113 of the transfer and preparation device 101 includes a mating feature 117 configured to mate with the sample transfer vial 190, which may be a thread-like connection or snap-fit connection.

Furthermore, in the embodiment of FIG. 1B, the preservative solution 104 is contained or packaged in the sample transfer vial 190 rather than the primary chamber 110 prior to use. Thus, utilization of temporary blocking membranes within the primary chamber 110 is unnecessary, and the upper end 111 of the transfer and preparation system 101 may be sealed by a removable upper cap 180 without a rupturing member (e.g., appendage 152). However, the upper cap 180 may still include the diaphragm 154 to facilitate flow of the cervical sample 102 through the transfer and preparation device 101.

In operation, the patient, upon collecting the cervical sample 102 with a personal cervical sample collection device 103, may transfer the cervical sample 102 from the cervical sample collection device 103 to the sample transfer vial 190. For example, the patient may swirl or rinse a tip of the cervical sample collection device 103 containing the cervical sample 102 in the preservation fluid 104. In certain embodiments, upon mixing of the cervical sample 102 with the preservation fluid 104, the patient may simply attach the transfer and preparation device 101 to the sample transfer vial 190 and send the transfer and preparation device 101 to a POC testing center or laboratory for further processing. In such embodiments, a clinician or lab technician, upon receipt of the transfer and preparation device 101, may transfer the mixed cervical sample 102 to the primary chamber 110, attach the removable upper cap 180, and press the diaphragm 154 to flow the mixed cervical sample 102 through the transfer and preparation device 101 to complete pre-processing prior to sample analysis. In certain other embodiments, upon mixing of the cervical sample 102 with the preservation fluid 104, the patient may utilize a disposable transfer pipette or other suitable device to transfer the mixed cervical sample 102 to the primary chamber 110 for pre-processing before sealing and sending the transfer and preparation device 101 to a POC testing center or laboratory for analysis.

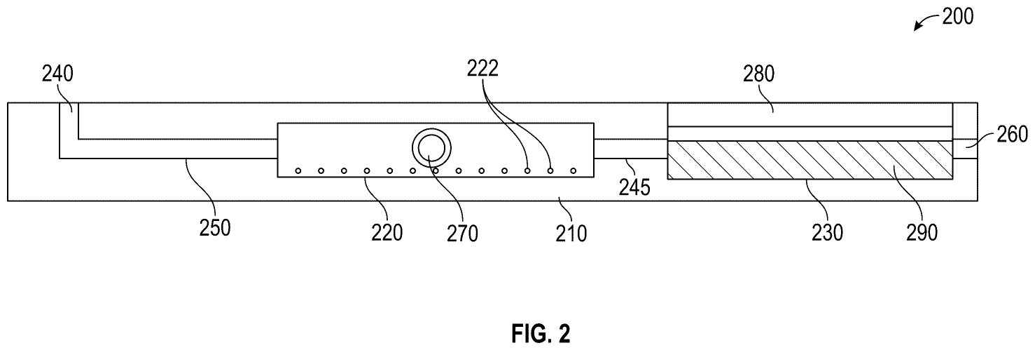

FIG. 2 illustrates a schematic diagram of an exemplary analysis cartridge 200, according to certain embodiments. The exemplary analysis cartridge 200 may be utilized in combination with other devices and methods of the present disclosure, such as the sample transfer and preparation devices 100 and 101.

The analysis cartridge 200 includes a frame 210 housing a primary reaction well 220 and a detection well 230 therein. The frame 210 is generally formed of a rigid or semi-rigid material such as plastic, cardboard, and the like. In certain embodiments, an outer surface of the frame 210 may include machine-printed barcodes (not shown) for manufacturing and/or patient identification information which may be fixed or transferrable utilizing a transferable film. A port 240 in the frame 210 enables engagement of the analysis cartridge 200 with a connection port (e.g., the connection port 170) of the transfer and preparation devices 100 and 101. Lysed cervical sample 102 is first introduced into the primary reaction well 220 via an origin conduit 250 fluidly coupled between the port 240 and the primary reaction well 220 and is flowed therefrom to the detection well 230 via the intermediate conduit 245. A detection window 280 disposed over the detection well 230 for detection of a signals produced by the target biomarkers via the human eye or with an analyzer, which may be located at the POC testing center or laboratory. The remaining flow-through of the cervical sample 102 is then flowed out of (e.g., removed therefrom) the detection well 230 by an evacuation conduit 260. In certain embodiments, the evacuation conduit 260 is fluidly coupled to the detection well 230 on a side opposing that of the intermediary conduit 245. In certain examples, a filter may be disposed in either the port 240 or the origin conduit 250 and configured to filter unwanted nucleic acids from the lysed cervical sample 102. The primary reaction well 220 is further fluidly coupled with a processing feed conduit 270 for introduction and removal of required solutions to aid in sample washing, signal development, and the like.

The primary reaction well 220 includes one or more unfixed primary substrates 222 having surfaces thereof modified or coated with capture and/or detection components (e.g., antibodies) against target biomarkers (e.g., target biomarkers or target biomarker complexes) in the lysed cervical sample 102. Suitable substrates 222 include any unfixed and solid-state substrates displaying chemical groups susceptible to reaction or modification for binding with capture and/or detection components specific for target biomarkers, such as non-conjugated antibodies recognizing cell surface molecules present on squamous epithelial cells. For example, in certain embodiments, the primary reaction well 220 includes a plurality of substrate beads 222 disposed on a bottom surface thereof, such as microbeads composed of metals (e.g., gold, iron, and cobalt, with or without magnetic properties), glass, silicas, and polymers (e.g., natural such as sepharose or synthetic such as polystyrene). The utilization of substrate beads 222 facilitates lateral flow type chromatographic analysis with the analysis cartridge 200, whereby target antigens in the cervical sample 102 are first captured on the primary substrates 222 in the primary reaction well 220, and then chromatographic flow is initiated via interaction with a secondary cartridge substrate 290 in the detection well 230 (discussed below). It should be noted that examples of capture components that may be coated onto the primary substrates 222 are not limited to antibodies, and may further include aptamers, nucleic acids, affibodies, aptabodies, proteins, peptides, and the like, which bind with strong affinity to target biomarkers.

Similar to the primary substrates 222, the secondary cartridge substrate 290 in the detection well 230 is also modified to interact with target biomarkers and facilitate creation of detection signals for analysis. In certain embodiments, the cartridge substrate 290 is a chromatographic membrane formed of, but not limited to, nitrocellulose, silica, paper, micro-patterned silicon, or polymeric materials. The properties of the cartridge substrate 290 may facilitate the capillary mobility of target biomarkers, such as proteins or microbead complexes in solution, towards a detection region having one or more detection surface molecules (e.g., binding agents) conjugated therewith. In certain embodiments, the cartridge substrate 290 is formed from a conductive polymer disposed on individual electrodes or an electrode array for electrochemical analysis of the cervical sample 102.

Generally, surfaces of the secondary cartridge substrate 290 are coated or modified with surface molecules that bind with different regions of target biomarkers and/or are capable of being modified with molecules reactive to the target biomarkers. For example, surface molecules that may be coated onto the cartridge substrate 290 include aptamers, nucleic acids, antibodies, affibodies, aptabodies, proteins, peptides, and the like, which bind with strong affinity (low K_{D} value) to target biomarkers. Further description of surface molecules that can be utilized in combination with the cartridge substrate 290 or primary substrates 222 are described below with reference to FIGS. 3-5.

In operation, the connection port 170 of the sample transfer and preparation system 100 or 101 is engaged with the port 240 of the analysis cartridge 200 for transfer of the lysed cervical sample 102 to the primary reaction well 220. In certain embodiments, the cervical sample 102 is diluted after lysis and before being flowed into the analysis cartridge 200 so as to prevent the lysis reagents 122 from inhibiting binding target biomarkers to the substrates in the analysis cartridge 200. For example, the lysed cervical sample 102 may be flowed through a dilution chamber (not shown) in the transfer and preparation system 100 or 101 located between the lysis chamber 120 and the connection port 170. In certain embodiments, this dilution is performed as the lysed cervical sample 102 is flowed into the analysis cartridge 200.

In certain embodiments, the analysis cartridge 200 is already disposed within an analyzer prior to engagement with the sample transfer and preparation system 100 or 101. In certain other embodiments, the analysis cartridge 200 is transferred to an analyzer after engagement with the sample transfer and preparation system 100 or 101. Target biomarkers, now released from the target cells in the cervical sample 102 (e.g., by lysis), are flowed through the port 240 and into the origin conduit 250 towards the primary reaction well 220. Unwanted nucleic acids and other cellular components may be trapped by filter(s) disposed in the port 240 or the origin conduit 250, while the remainder of the cervical sample 102 is flowed into the primary reaction well 220 for capture of target biomarkers on the primary substrates 222. The primary substrate-bound biomarkers are then flowed into the detection well 230 and are captured by the secondary cartridge substrate 290, which includes surface molecules for capturing the already-bound target biomarkers in the cervical sample 102. The bound biomarkers are then washed, developed, and/or labeled for subsequent analysis with the analyzer, which detects one or more signals facilitated by the binding of the target biomarkers with the primary substrates 222 and/or secondary cartridge substrate 290. Depending on the type of primary substrate 222 utilized, accumulating detection signals (e.g., simple colors) can be visualized by eye or with a sensor as a function of the primary substrate 222.

FIG. 3 illustrates an exemplary binding arrangement of capture and detection molecules conjugated on the secondary cartridge substrate 290. As illustrated in FIG. 3, the capture and detection molecules include at least a capture molecule 310 that is configured to “sandwich” a target biomarker (e.g., antigen) 302 along with a detection molecule 320 conjugated to a primary substrate 222 and/or an enzyme-linked label 322. In certain embodiments, the detection molecule 320 is a capture component (e.g., molecule) conjugated to the surface of the primary substrates 222 in the primary reaction well 220, and may react with the target biomarker 302 prior to binding of the target biomarker with the capture molecule 310 immobilized on the secondary cartridge substrate 290. In certain embodiments, the detection molecule 320 is added to the detection well 230 unconjugated and in solution, and may react with the target biomarker 302 before or after binding of the target biomarker 302 with the capture molecule 310. In certain other embodiments, the capture molecule 310 is added to the detection well 230 in solution, and may react with the target biomarker 302 before or after binding of the target biomarker 302 with the detection molecule 320.

In embodiments where the label 322 is utilized, the label 322 generally includes any suitable type of stain, tag, dye, or conjugate to visualize the captured target biomarkers 302. For example, the label 322 may include an enzyme, a fluorophore, or an electrochemiluminescent tag, depending on the surface molecules and the type of immunoassay technique utilized. In certain embodiments, the label 322 includes a streptavidin-horseradish peroxidase (HRP) conjugate having a high affinity for biotin, and therefore may be utilized in combination with a biotinylated detection molecule 320 such as a biotinylated antibody to detect target biomarkers 302. In such embodiments, the secondary cartridge substrate 290 may further include any suitable chromogenic substrates configured to react with HRP and enable HRP-mediated production of signal species during immunoanalysis, including 3,3′-diaminobenzidine (DAB), 3,3′5,5′-tetramethylbenzidine (TMB), 2,2′-Azinobis [3-ethylbenzothiazonline-6-sulfonic acid] (ABTS), or o-pheylenediamine dihydrochloride (OPD). Other suitable enzyme reporters and chromogenic substrates include alkaline phosphatases used in combination with nitro blue tetrazolium chloride (NBT) and 5-bromo-4-chrloro-3-indoyl phosphate (BCIP) or p-nitrophenyl phosphate (PNPP), glucose oxidase used in combination with NBT, and beta-galactosidase used in combination with 5-bromo-4-chloro-3-indoyl-B-d-galactopyranoside (BCIG or X-Gal).

In embodiments without the utilization of an enzyme-linked label 322, reporting may be a function of the primary substrate 222 already conjugated with the target biomarker 302, or a molecule that is part of the complex between target biomarker 302 and the primary substrate 222.

Examples of suitable types of surface molecules that may be utilized as capture molecules 310 and detection molecules 320 include aptamers, nucleic acids, antibodies, affibodies, aptabodies, proteins, peptides, and the like, which bind with strong affinity to desired target biomarkers 302. In certain embodiments, the capture and/or detection molecules 310, 320 include surface molecules specific to viral and cellular protein epitopes of target biomarkers 302, which may be oncoproteins from high-risk strains of HPV. For example, the capture and/or detection molecules 310, 320 may include antibodies specific to E6 and/or E7 oncoproteins from three or more hr-HPV strains (e.g., isoforms) including HPV16, HPV18, HPV31, HPV33, HPV35, HPV36, HPV45, HPV51, HPV52, HPV56, HPV58, HPV59, among others. In certain embodiments, the capture and/or detection molecules 310, 320 include antibodies specific to E6 and/or E7 oncoproteins from up to 12 hr-HPV strains. In certain other embodiments, the capture and/or detection molecules 310, 320 include antibodies specific to E6 and/or E7 from twelve or more hr-HPV strains. As described above, the presence of HPV E6 or E7 oncoproteins in a cervical sample indicates high-risk, advanced HPV infections and cervical disease since E6 and E7 are expressed by HPV following infection, host cell genomic integration, and loss of negative viral regulators.

In addition to antibodies for hr-HPV oncoproteins, the capture and/or detection molecules 310, 320 include molecules specific for epitopes of cellular proteins whose levels may be altered by HPV oncoproteins such as HPV E6 and/or E7, and/or cellular proteins which provide sample adequacy or disease indications independent of HPV oncoproteins. For example, the capture and/or detection molecules 310, 320 may include antibodies specific for cellular keratin (i.e., cytokeratin) proteins that are present in cervical samples, such as cytokeratin proteins K5, K8, K17, and K18. Levels of cytokeratin proteins K5, K8, and K18 do not change as a function of HPV infection or cervical disease, and therefore K5, K8, and K18 provide evidence of sample viability and adequacy. Cytokeratin protein K17 provides diagnostic and prognostic information for cervical samples positive for cervical disease independent of HPV infection, such as high grade lesions and cervical carcinomas. Therefore, utilizing antibodies for E6 and/or E7 oncoproteins in combination with antibodies for cytokeratin proteins (e.g., K5, K8, K18, and K17) enables a more complete analysis of cervical disease with improved detection sensitivity in a single rapid screen, reflecting not only evidence of HPV infection, but also the level of cervical disease. Accordingly, the multiplexed approach described herein reduces the amount of tests necessary to sufficiently and accurately screen for cervical disease, facilitating access to lower resource areas with the greatest need for such screening. Although cytokeratins K5, K8, K17, and K18 are described above, it is further contemplated that additional cytokeratins, such as cytokeratins K4, K6, K10, and K13, may be utilized as biomarkers during analysis of cervical sample 102.

In certain embodiments offering quantitative detection, the capture and detection molecules further include capture and/or detection molecules 310, 320 specific for epitopes of beta-actin or glyceraldehyde 3-phosphate dehydrogenase (GAPDH). Beta-actin plays a critical role in most cellular processes, including cell migration, cell division, and gene expression regulation, while GAPDH is integral in glycolysis. Like cytokeratins K5, K8, and K18, levels of beta-actin and GAPDH are not affected by cervical disease status. The stable and ubiquitous expression of these proteins thus makes them suitable sample control biomarkers to normalize the sample to sample variability when quantitating other cellular markers or assessing the quality of a cervical specimen.

FIG. 4A-4B illustrate tables 400 and 450 with additional target biomarkers 302 that may be screened with the biomarkers discussed above, according to embodiments of the present disclosure. Table 400 includes target biomarkers 302 listed individually, while table 450 includes the target biomarkers 302 listed in exemplary multiplexed panels. The exemplary target biomarkers 302 include proteins whose levels are altered by HPV oncoproteins and indicate the presence of cervical intraepithelial neoplasia 2 (CIN2) or 3 (CIN3), such as E-cadherin, ERK-1, LR67, MMP-2, NF-κB, nm23-H1, P16^{INK4a}, PCNA, survivin, hTERT, Topo-2α, and VEGF-C.

As shown in FIG. 4A, table 400 also provides the subcellular localization of each biomarker as well as expression events related to CIN3 and/or cervical cancer. Table 450 in FIG. 4B, on the other hand, provides the relative sensitivity, specificity, positive predictive value (PPV), negative predicative value (NPV), receiver operator characteristics (ROC), and odds ratio (OR) of the multiplexed target biomarkers 302 in an original biopsy setting. The inventors have surprisingly found that multiplexing of the target biomarkers 302 resulted in increased sensitivity and PPV thereof, particularly when combining markers at the histological level.

In addition to the biomarkers described above with reference to FIGS. 4A-4B, it is further contemplated that target biomarkers 302 may also include any suitable molecules such as, but not limited to proteins, nucleic acids, lipids, carbohydrates, and the like, having viral, bacterial, fungal, parasitic, or host cell origin.

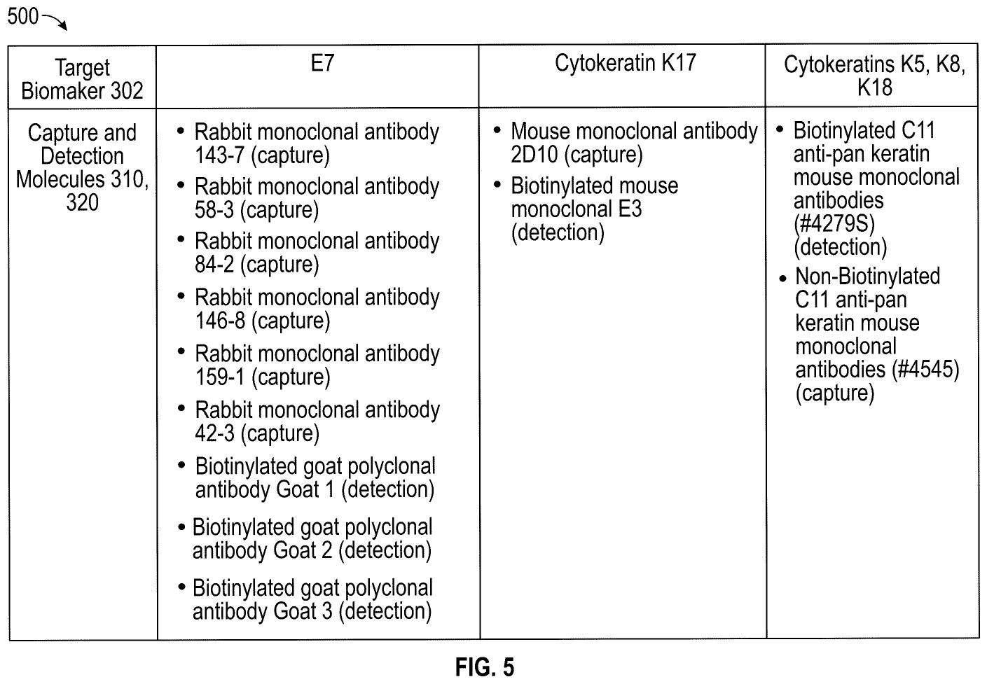

FIG. 5 depicts a table 500 of exemplary antibodies that may be utilized as capture and/or detection molecules 310, 320 to bind to target biomarkers or antigens 302, in accordance with aspects of the present disclosure. The multiplexed detection and analysis of cervical disease biomarkers described herein may utilize combinations of different monoclonal and polyclonal antibodies isolated from various sources. As shown in FIG. 5, exemplary antibodies include monoclonal rabbit antibodies and polyclonal goat antibodies reactive with different epitopes of HPV E7 oncoproteins, monoclonal mouse antibodies reactive with cytokeratin K17, as well as anti-pan keratin monoclonal mouse antibodies reactive with cytokeratin K5, K8, and K18. Although table 500 includes antibodies for E7 and cytokeratins K5, K8, K18, and K17, it is further contemplated that the capture and detection molecules utilized for detection and analysis of cervical samples 102 may include aptamers, nucleic acids, affibodies, aptabodies, proteins, peptides, and the like. Additionally, the capture and detection molecules may include surface molecules specific to other target antigens useful as biomarkers for cervical disease, such as survivin, TERT, ERK, and other biomarkers having sensitivity and specificity for CIN2+, as described above.

In certain embodiments, capture molecules 310 for oncoprotein E7 include rabbit monoclonal antibodies 143-7, 58-3, 84-2, 146-8, 159-1, and/or 42-3, which may be used in any suitable combinations thereof. Detection molecules 320 for oncoprotein E7 include biotinylated goat polyclonal antibodies Goat 1, Goat 2, and/or Goat 3, which may be used in any suitable combinations thereof along with the capture molecules 310 above to sandwich oncoprotein E7 on the cartridge substrate 266 for detection and analysis. Generally, detection of E7 from at least HPV strains 16, 18, and 45 requires at least two rabbit monoclonal antibodies and at least two goat polyclonal antibodies to achieve optimum detection. For example, in certain embodiments, a 1:1 combination of rabbit monoclonal antibodies 42-3 and 143-7 is utilized for capture of E7 from HPV16, HPV18, and HPV45, and a 1:1 mixture of biotinylated goat 1 and biotinylated goat 2 is utilized for detection thereof. Detection of E7 from a greater number of strains, such as all twelve hr-HPV strains, requires at least five rabbit monoclonal antibodies and at least three goat polyclonal antibodies for optimum detection. For example, in certain embodiments, a combination of rabbit monoclonal antibodies 143-7, 58-3, 84-2, 146-8, and 159-1 is utilized for capture of E7, along with a 1:1:1 mixture of biotinylated goat polyclonal antibodies 1, 2, and 3 for detection.

As further depicted in the table 500, in certain embodiments, the capture molecules 310 for cytokeratins K5, K8, and K18 include non-biotinylated C11 anti-pan keratin mouse monoclonal antibodies, while the detection molecules 320 include biotinylated C11 anti-pan keratin mouse monoclonal antibodies. In certain embodiments, the capture molecules 310 for cytokeratin K17 include the mouse monoclonal 2D10 antibody, while the detection molecules 320 include biotinylated mouse monoclonal E3 antibodies. The antibodies described above provide optimal reactivity with desired cytokeratins for improved detection by the analyzer, described below in greater detail.

Detection of target biomarkers 302 may be carried out by a benchtop analyzer at a POC testing center or laboratory configured to accept and process the analysis cartridges (e.g., analysis cartridge 200) described above. The benchtop analyzer is generally configured to carry out immunoanalysis of target biomarkers 302 isolated from the cervical sample 102, such as by chemiluminescence immunoassay (CLIA), enzyme-linked immunosorbent assay (ELISA), radioimmunoassay, counting immunoassay, fluoroimmunoassay, and the like. Generally, the immunoassays described herein involve comparison of the concentrations (e.g., levels of expression) of the target biomarkers 302 in the cervical sample 102 with a control or reference sample utilizing one or more predetermined algorithms programmed into the analyzer. In certain embodiments, the analyzer is designed to facilitate reverse immunochromatography analysis of the cervical samples 102 along a substrate within the analysis cartridges, such as cartridge substrate 266. Immunochromatography processing parameters, such as sample reaction times, fluid input and output, and fluid volumes are controlled and executed directly by the analyzer. In such embodiments, the analyzer detects the presence of immune complexes within the defined regions (e.g., contrast and detection regions) of the analysis cartridge and automatically dispenses the necessary chemistries of washing solutions, developing solutions, stop solutions, and the like for reverse chromatographic processing. The analyzer further executes analysis of the quantitative signals obtained from the contrast and detection areas for reporting target biomarker 302 levels (e.g., concentrations) to a user via comparison of sample test results with internal standard curves, which may be displayed on a display screen of the analyzer.

Quantitative and qualitative data produced from analysis of the cervical samples 102 may be collected and stored on the analyzer or an external storage device (e.g., computer) for physician and/or patient retrieval and use. In certain embodiments, the analyzer or external storage device is configured to transfer or transmit data to an external device in direct or indirect communication with the analyzer, such as a Bluetooth-enabled wireless communications device, thus enabling the performance of telemedicine or telehealth. Data transferred from the analyzer may be viewed on a mobile interface application configured to synchronize and display data for a user thereof. In certain embodiments, the mobile interface application is further configured to facilitate hardware and software updates, as well as calibration, telemedicine, data processing, and data transfer (e.g., to patients, physicians, providers, etc.).

In summary, certain embodiments of the present disclosure include devices and methods of improved screening for cervical disease, and in particular, improved devices and methods for multiplexed biomarker screening of self-collected cervical samples. The utilization of a self-collector and sample transfer vial enable collection of personal cervical samples from a patient's own home, while the multiplexed biomarker panels described herein provide sufficient information with respect to presence of cervical disease to eliminate the need for subsequent confirmative testing. Accordingly, the devices and methods described herein reduce the time and costs associated with conventional cervical disease screening and enable expansion of cervical screening to both developed regions with existing programs as well as lower resource areas with the greatest need.

### Nucleic Acid-Based Screening for HPV-Associated Cervical Disease

The present disclosure further relates to methods, devices, and systems for determining evidence of disease resulting from infections with high-risk human papillomavirus (HPV), such as cervical intraepithelial neoplasia (CIN) and/or cervical cancer in liquid-based cervical specimens or certain anal or head and neck cancers, using rapid, reverse transcriptase, polymerase chain reaction (RT-PCR) of RNA biomarkers at the point-of-care (POC).

The present disclosure provides a POC RT-PCR method for the rapid detection of critical mRNA/DNA biomarkers reflective of HPV, CIN, and/or cervical cancer, as well as disease progression and prognostic risk. This approach addresses deficiencies in both time-to-results, as well high resource demands, that inhibit the implementation of cervical screening programs in developing countries. Furthermore, this approach improves upon the accuracy and clinical utility of screening by providing objective metrics as compared to subjective metrics, and simultaneously defining the at-risk population of HPV-positive women who carry high-risk infections of HPV that are most likely to advance to cervical neoplasia/cancer.

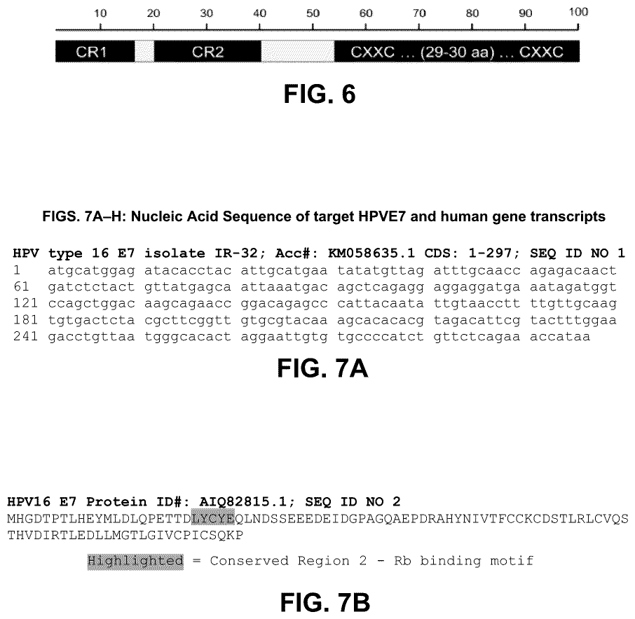

In certain embodiments, an RT-PCR assay determines the presence of an oncogenic biomarker HPVE7 by detecting mRNA transcripts in real-time, qualitative for semi-quantitative fashion for the determination of cervical disease. HPV-E7 is an important, highly conserved oncoprotein, whose presence is elevated upon integration of viral DNA into the host genome. The oncoprotein is critical for HPV-induced carcinogenesis and has been shown to interact with key host cell factors such as pRb, disrupting normal function (Cell. 2017 Sep. 7; 170 (6): 1164-1174.e6; doi: 10.1016/j.cell.2017.08.001). Analysis indicates the E7 sequence exhibits hypovariation compared to other HPV genes, including the oncogene E6, representing one of the most constrained HPV genes and highlighting its critical contribution to cellular transformation. An example of the conserved regions of HPVE7 oncoproteins is provided in FIG. 6.

In certain embodiments, an RT-PCR assay employs a multiplex panel of nucleic acid biomarkers including representative sequences of high-risk HPV, as well as host cell factors k17, survivin, and/or others at the POC to detect indication of HPV infection, the development of HPV-related disease or transformation, disease progression, and/or survival risk in a single test.

The methods, devices, and systems disclosed herein include an automated, preassembled cartridge, with portable instrumentation for a robust, hands-off approach providing an opportunity to deliver a POC assay for HPV-related disease that can be operated in low resource environment, with high sensitivity and specificity. Enzymes and reagents, along with unique primer sets, may be preassembled within the single use cartridges to facilitate automated instrument-controlled processing for all required stages, including mRNA extraction, reverse transcriptase-mediated cDNA construction, target sequence amplification, and detection of specific HPV and cellular nucleic acid biomarkers indicative of HPV induced disease, progression, and/or risk.

In certain embodiments, the methods, devices, and/or systems disclosed herein enable the accurate and rapid identification of HPV-positive women who are at the greatest risk for the development of cervical cancer, via a low cost, rapid assay administered at the point-of care. Using specific RNA transcripts produced by the virus as well as host cell genomes, the methods and devices identify patients with persistent, high-risk strains of HPV that have initiated critical steps associated with cellular transformation and development of HPV-related disease. The methods and devices employ an RT-PCR assay configured within single use cartridges designed to facilitate automated processing for all required stages, including mRNA extraction, reverse transcriptase-mediated cDNA construction, amplification, and detection of specific HPV and cellular biomarkers indicative of HPV induced disease, progression and/or risk. The assay relies on a low-cost and robust tool approach to rapidly detect specific viral and/or RNA transcripts that reflect both the presence of persistent, integrated, high-risk strains of HPV as well cervical disease in a single test.

The assay is designed and suitable for use with a variety of specimens including saliva, blood, swabs, biopsies, etc. In certain embodiments, the assay accepts liquid based cervical specimens, which may have been collected into cytological, preservative buffers by a trained professional, or collected directly by the patient into a suitable specimen buffer/preservative using an at-home cervical sample self-collector.

Although embodiments disclosed herein are described with reference to cervical disease, high-risk strains of HPV are also causative of other anogenital as well as head and neck cancers, indicating that a rapid assay for HPV DNA/RNA may be beneficial beyond the field of cervical cancer. Although the sensitivity for CIN2+, CIN3+ and cervical carcinoma has been observed in ELISA-based protein assays for HPVE7 to be ˜36%, 58% and 86%, respectively, the specificity and negative predictive values for E7 are roughly 98%-99.9%, demonstrating its critical association with subsequent cancer development. The performance and application of HPVE7 mRNA as an indicator of CIN and cancer may be further improved by use of an RT-PCR assay that detects multiple targets including HPV L1 DNA, as well as other viral and host cell mRNA biomarkers thereby reporting upon both the presence of high-risk strains of HPV as well as the presence and progression of disease in a single assay.

Current methods for cervical screening require both an HPV DNA assay and subsequent reflex cytology to reach similar clinical endpoints. The format presented here is particularly suitable for low resource areas, where HPV-related disease and death predominates, minimizing sample preparation and run times, without the demands of typical laboratory infrastructure lacking in low resource regions.

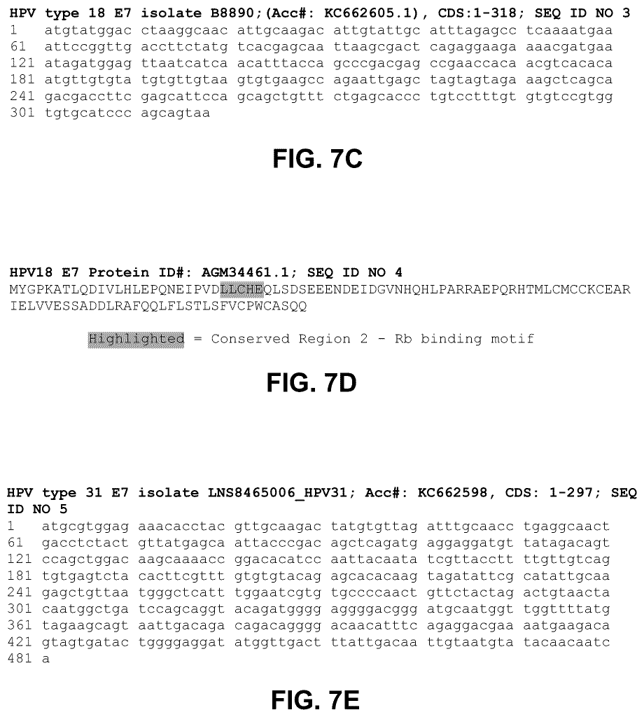

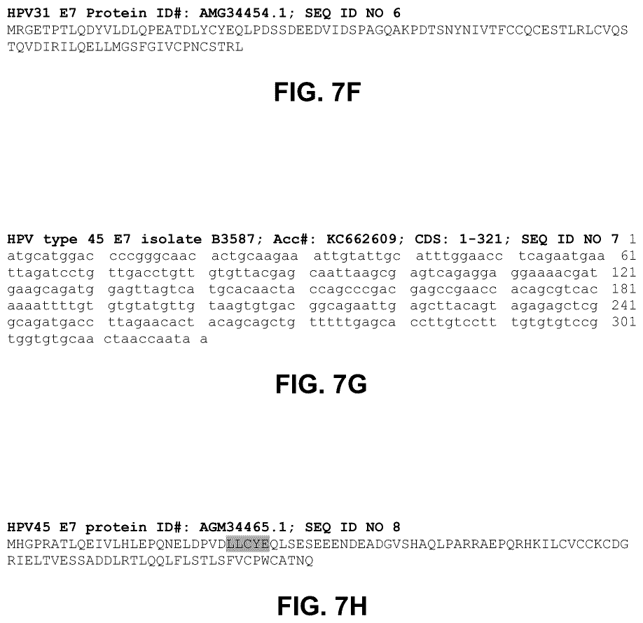

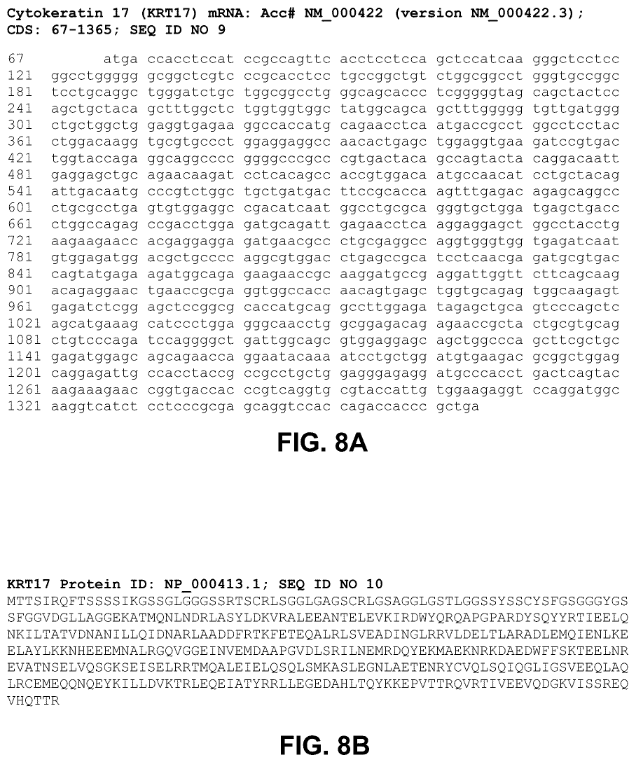

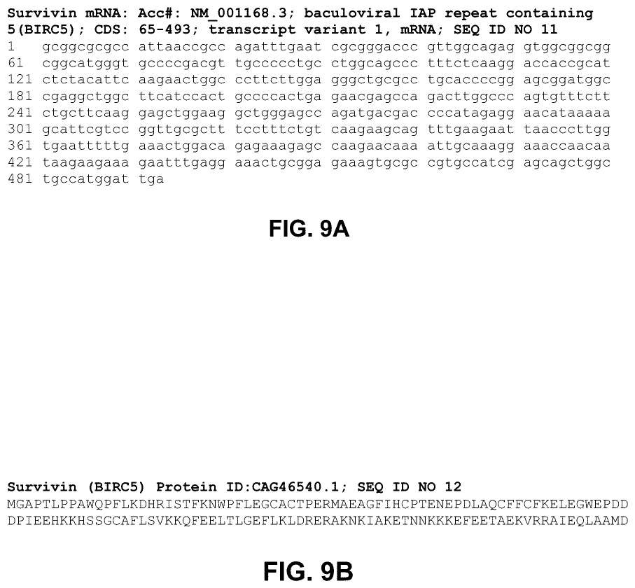

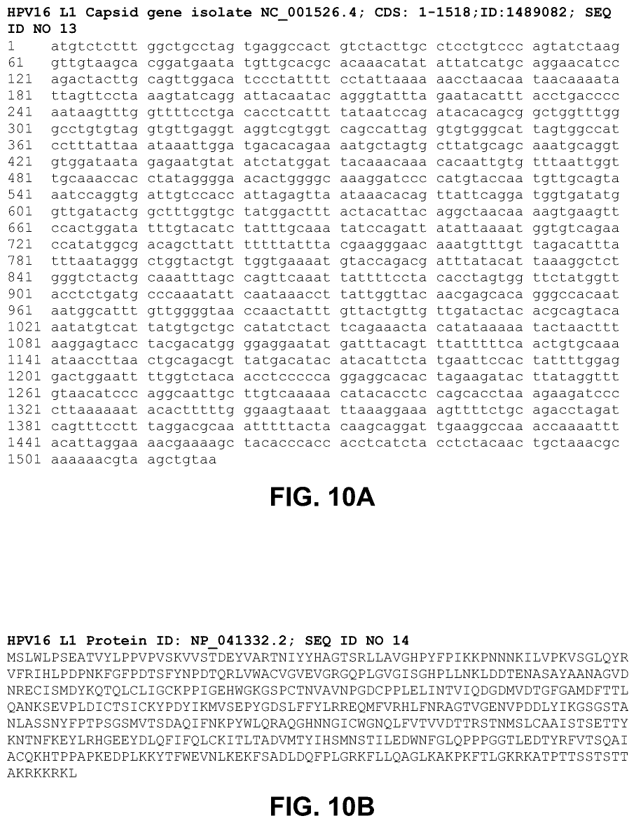

In certain embodiments, the assay utilizes specific primers to recognize and amplify target RNA species originating from viral or host cell sources. In the case of HPV-E7 RNA, the transcript exhibits highly conserved sequences, shared amongst several high-risk strains responsible for the majority of cervical cancers observed world-wide. Primers may be directed toward these conserved regions provided they are specific for high-risk strains of HPV-E7. Nucleotide sequences of several representative target species are illustrated in FIGS. 6-10.

In certain embodiments, a cervical specimen is collected directly by the patient, clinical personnel, or physician etc., using a self-collection device (e.g., iPap Personal Sample Collector commercially available from OncoGenesis, Inc. in Salt Lake City, UT) and placed into a liquid preservative buffer for preservation of molecular target species and inactivation of infectious material, rendering the specimen safe and preparing it for subsequent analysis. A sample of the specimen is introduced into the reaction cartridge where upon it mixes with RT-PCR reagents in distinct chambers to initiate sample processing, amplification and detection. Uniquely designed primer chemistry sets provide target recognition for cDNA conversion and amplification, facilitating amplification and detection of target RNA species. Detection can be accomplished using a variety of indicators including molecular probes, fluorescent dyes/labels, pH indicator dyes, etc. Preferred embodiments employ visible fluorescent signaling but may utilize colorimetric, and/or redox signaling, either of which is collected and processed by the cartridge-related instrument. The instrument supports all requirements of the cartridge (power, valve activation, temperature controls, signal collection and processing, etc.) and is configured for single or multi-cartridge use.

Preferred target mRNA species for the invention include the highly conserved oncogene E7 expressed by high-risk HPV strains and critical for cell transformation, as well as a panel of mRNA transcripts for host genes such as but not limited to survivin, KR17, p16 etc., whose products function within pathways directly influenced by viral oncoproteins, or which have been shown to be associated with lower survival in patients positive for cervical cancer. Preferred target DNA for demonstration of HPV presence currently include the gene for HPV L1 capsid protein from high-risk strains but may include other DNAs indicative and selective for high-risk HPV.

In certain embodiments, the assay utilizes unique co-primer sets to amplify HPV mRNA encoding E7 from high-risk strains, such as HPV16, HPV18, HPV31, and HPV45, demonstrating evidence of viral integration and upregulation of a major viral oncoprotein critical for carcinogenesis. Additionally, assay designs will include the targeted amplification and detection of HPV DNA of high-risk strains using primers directed at the L1 or other viral specific DNA gene or region selective for high-risk HPV strains. In this manner the assay in its simplest configuration reports upon both the presence of high-risk HPV DNA (conferring high sensitivity) as well as mRNA for an HPV-related disease biomarker E7 (high specificity) in a single assay. Multiplex embodiments include a panel of mRNAs of host cell gene expression indicated above, providing further clinical evidence of cervical neoplasia, disease progression, and survival risk, improving the degree of clinical information that can be gleaned from a single assay at the POC.

In summary, the certain embodiments of the present disclosure describe a reverse-transcriptase polymerase chain reaction (RT-PCR) assay/method preassembled within a single use disposable, cartridge to screen patient cervical specimens for nucleic acid biomarkers indicative of HPV-related cervical disease, progression and/or cervical cancer, and survival risk at the point of care (POC). Automated, single use cartridges are supplied preassembled and loaded with necessary components for automated, rapid processing using a robust, portable instrument. The cartridge is designed to carry out all steps of RT-PCR procedure from RNA extraction through cDNA conversion, target amplification, detection and results reporting. The assay takes advantage of specifically designed primer sets, which facilitate the detection of viral nucleic acids indicative of high-risk HPV infections, as well nucleic acid biomarkers reflecting expression of host-cell genes, providing objective evidence of disease progression and cancer risk at the POC. The test can be achieved with minimal, low-cost hardware, and may be detected using visible, fluorescent or redox means, and can be completed in rapid fashion (<30 minutes) with minimal sample manipulation or technical expertise, suitable for use in low resource environments. The assay provides objective results at the point of care for multiple clinical endpoints including evidence of high-risk HPV infections, disease progression and/or cancer survival risk in a single, low-cost test, instead of subjective or multiple, expensive testing procedures such as cytology and HPV DNA screening, reducing both the time and expense associated with typical cervical screening methods.

While the foregoing is directed to embodiments of the present disclosure, other and further embodiments of the disclosure may be devised without departing from the basic scope thereof, and the scope thereof is determined by the claims that follow.

### EXAMPLE EMBODIMENTS

Embodiment 1: A method (design and application) of a RT-PCR assay for the rapid detection of CIN and/or cervical cancer at the point-of care comprising contacting a patient's biological sample with a of a plurality of reagents specific for the binding and amplification of target nucleic acid species RNA/DNA reflecting cervical disease, including at least one viral oncoprotein from high-risk strains of HPV that is elevated following viral integration into the host genome.

Embodiment 2: The method of Embodiment 1 described above, whereby the amplification of target RNA biomarkers for cervical cancer is detected using visible colorimetric, fluorescent or redox means.

Embodiment 3: The method of Embodiment 2 described above, in which the visible detectable colorimetric change is produced by a pH indicator present in the reaction.

Embodiment 4: The method of Embodiment 2 described above, in which the fluorescent signal is generated by virtue of inclusion of a fluorescent probe, or tag and the redox signal is generated by inclusion of redox enzyme(s).

Embodiment 5: The method of Embodiment 1 described above, in which the biological sample is a cervical specimen in a buffer that inactivates infectious agents while preserving targets and preparing the specimen for subsequent molecular analysis.

Embodiment 6: The method of Embodiment 1 described above, in which the preferred target RNA biomarker(s) include the specific amplification of viral RNA transcripts for HPVE7 protein from at least one high-risk strain associated with cervical disease.

Embodiment 7: The method of Embodiment 1 described above, in which the preferred target RNA biomarker(s) includes the specific amplification of viral RNA transcripts for HPVE6 protein from at least one high-risk strain associated with cervical disease.

Embodiment 8: The method of Embodiment 1 described above, in which an RNA target for amplification includes a prognostic factor such that it demonstrates evidence of cancer survival risk.

Embodiment 9: The method of Embodiment 1 described above, in which the prognostic factor is represented by mRNA transcripts for cytokeratin K17.

Embodiment 10: The method of Embodiment 1 described above, in which the target biomarkers for amplification and detection include at least one RNA transcript for viral oncoproteins and one RNA transcript from host genome reflective of disease and/or progression or prognostic risk.

Embodiment 11: The method of Embodiment 1 described above, in which the target biomarkers for amplification include DNA sequence from high-risk strains of HPV.

Embodiment 12: The method of Embodiment 11 described above, in which the target DNA sequence for amplification is represented by the L1 capsid gene for HPV.

Embodiment 13: The method of Embodiment 12 described above, in which the target DNA sequence for L1 is shared amongst high-risk strains of HPV and not low risk viral strains.

Embodiment 14: The method of Embodiment 5 described above, in which the biological specimen is an anal swab or biopsy of suspected anal lesions.

Embodiment 15: The method of Embodiment 5 described above, in which the biological specimen is a sample of suspected head or neck cancer/lesion.

Embodiment 16: The method of Embodiment 6 described above, in which the primers for amplification and detection of viral oncogenes are designed against conserved sequences found in HPV E7 mRNA and/or HPV LI DNA from high-risk strains for HPV, and primers for amplification and detection of expression of host cell genes are designed against a panel or subset of human mRNAs, including but not limited to cytokeratin K17, survivin (BIRC-5), and/or ERK, p16, VEGF-c, hTERT, NF-kB, E-cadherin, LR67, PCNA, or Topo2-alpha.

Embodiment 17: A device for automated processing and amplification of a biological sample, comprising: a single use cartridge preloaded with reaction chemistry, primers, etc.

Embodiment 18: The device of Embodiment 17 described above, in which the cartridge is bar-coded to establish testing parameters for instrument, patient ID and results record.

Embodiment 19: The device of Embodiment 17 described above, in which any power, valve activation, temperature changes, mechanics, etc., required for function/processing are supplied by an instrument configured to accept and process cartridge.

Embodiment 20: The device of Embodiment 19 described above, in which the instrument is designed to accept one or more cartridges for simultaneous processing.

Embodiment 21: The device of Embodiment 20 described above, in which data is electronically stored and transferable to portable or other handheld devices such as phone, computer, tablet, etc., via Bluetooth and/or wireless protocol.

Embodiment 22: The device of Embodiment 20 described above, in which the instrument includes optical and electronic components for signal collection as well as algorithm for signal processing is stored and activated for results calculation and reporting.

Embodiment 23: The device of Embodiment 20 described above, in which the instrument provides a fixed user interface or capability for control via hand-held device.

Embodiment 24: An instrument for performing rapid assay processing, the instrument configured to provide results in under 30 minutes.

Embodiment 25: The method of Embodiment 1 described above, which includes primers for the amplification of internal standard(s) for process and/or sample control.

Embodiment 26: The method of Embodiment 25 described above, further comprising a control which may include but is not limited to beta-globin, beta-actin, GAPDH, and/or cytokeratins 5, 8 or 18.

Embodiment 27: A point-of-care device for detecting cervical disease in a biological sample, comprising: a disposable cartridge having a chromatographic membrane formed of nitrocellulose, silica, paper, micro-patterned silicon, or polymeric materials, the chromatographic membrane further conjugated with a plurality of binding agents specific to: at least one human papillomavirus (HPV) oncoprotein from three or more high-risk HPV (hr-HPV) isoforms; at least one independent prognostic indicator for cervical disease; and at least one independent indicator of sample viability.

Embodiment 28: The device of Embodiment 27 described above, wherein the three or more hr-HPV isoforms include up to twelve hr-HPV isoforms comprising HPV16, HPV18, HPV31, HPV33, HPV35, HPV36, HPV45, HPV51, HPV52, HPV56, HPV58, and HPV59.

Embodiment 29: The device of Embodiment 27 described above, wherein the at least one independent prognostic indicator for cervical disease is cytokeratin K17.

Embodiment 30: The device of Embodiment 27 described above, wherein the at least one independent indicator of sample viability is selected from the group consisting of K4, K5, K6, K8, K13, and K18.

Embodiment 31: The device of Embodiment 27 described above, wherein the plurality of binding agents are further specific to one or more of E-cadherin, ERK-1, LR67, MMP-2, NF-κB, nm23-H1, P16INK4a, PCNA, survivin, hTERT, Topo-2α, and VEGF-C.

Embodiment 32: The device of Embodiment 31 described above, wherein the plurality of binding agents comprises one or more types of aptamers, nucleic acids, antibodies, affibodies, aptabodies, proteins, or peptides specific to epitopes of the HPV oncoprotein.

Embodiment 33: The device of Embodiment 32 described above, wherein the plurality of binding agents comprises a detector molecule conjugated to an enzyme, a fluorophore, or an electrochemiluminescent tag.

Embodiment 34: The device of Embodiment 33 described above, wherein the detector molecule is bound to a streptavidin-horseradish peroxidase (HRP) conjugate.

Embodiment 35: The device of Embodiment 27 described above, further comprising: an analyzer capable of electronic data transfer for external review of analysis results (telemedicine).

What is claimed is:

1. A method of detecting cervical disease via reverse transcription polymerase chain reaction (RT-PCR), comprising:
contacting a biological sample with a plurality of binding agents, the plurality of binding agents configured to bind to target nucleic acid species in the biological sample indicating cervical disease, the target nucleic acid species comprising:
at least one nucleic acid species from a viral oncoprotein of HPV;
binding the target nucleic acid species to the binding agents;
amplifying the target nucleic acid species via RT-PCR; and
detecting the amplified target nucleic acid species.

2. The method of claim 1, wherein the amplified target nucleic acid species are detected via colorimetric, fluorescent, or redox detection methods.

3. The method of claim 2, wherein the amplified target nucleic acid species are detected via colorimetric detection methods utilizing a pH indicator.

4. The method of claim 2, wherein the amplified target nucleic acid species are detected via fluorescent detection methods utilizing a fluorescent probe.

5. The method of claim 2, wherein the amplified target nucleic acid species are detected via redox detection methods utilizing one or more redox enzymes configured to produce a redox signal upon detection of the amplified target nucleic acid species.

6. The method of claim 1, wherein the biological sample comprises a cervical sample.

7. The method of claim 1, wherein the target nucleic acid species comprise target RNA biomarkers.

8. The method of claim 7, wherein the target RNA biomarkers include viral RNA transcripts from a HPVE7 protein or HPVE6 protein from at least one high-risk strain of human papillomavirus (HPV).

9. The method of claim 7, wherein the target RNA biomarkers include mRNA transcripts for cytokeratin K17 for demonstrating evidence of cancer survival risk.

10. The method of claim 1, wherein the target nucleic acid species comprise DNA sequences from high-risk strains of HPV.

11. The method of claim 10, wherein the DNA sequences comprise the L1 capsid gene for HPV.

12. The method of claim 1, wherein the biological specimen comprises an anal swab sample or a biopsy sample from an anal lesion.

13. The method of claim 1, wherein the biological specimen comprises a sample of a suspected head or neck cancer.

14. The method of claim 1, wherein the amplification and detection is performed using primers for at least one of HPV E7 mRNA or HPV LI DNA.

15. The method of claim 1, wherein the amplification and detection is performed using primers for at least one of cytokeratin K17, survivin (BIRC-5), and/or ERK, p16, VEGF-c, hTERT, NF-kB, E-cadherin, LR67, PCNA, or Topo2-alpha.

16. The method of claim 1, wherein the amplification and detection is performed using primers for at least one of beta-globin, beta-actin, GAPDH, cytokeratin 5, cytokeratin 8, or cytokeratin 18.

17. A device for automated processing and amplification of a biological sample, the device configured to:
contact a biological sample with a plurality of binding agents, the plurality of binding agents configured to bind to target nucleic acid species in the biological sample indicating cervical disease, the target nucleic acid species comprising:
at least one nucleic acid species from a viral oncoprotein of HPV;
bind the target nucleic acid species to the binding agents;
amplify the target nucleic acid species via RT-PCR; and
detect the amplified target nucleic acid species.

18. The device of claim 17, wherein the amplified target nucleic acid species are detected via colorimetric, fluorescent, or redox detection methods.

19. The device of claim 17, wherein the amplified target nucleic acid species are detected via colorimetric detection methods utilizing a pH indicator.

20. The device of claim 17, wherein the amplified target nucleic acid species are detected via fluorescent detection methods utilizing a fluorescent probe.


SEQUENCE LISTING (14 sequences)

SEQ ID NO: 1 - DNA, 297 bp, Human papillomavirus type 16
  misc_feature 1..297
  source 1..297: mol_type = other DNA
atgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccataa

SEQ ID NO: 2 - protein, 98 aa, Human papillomavirus type 16
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 3 - DNA, 318 bp, Human papillomavirus type 18
  misc_feature 1..318
  source 1..318: mol_type = other DNA
atgtatggacctaaggcaacattgcaagacattgtattgcatttagagcctcaaaatgaaattccggttgaccttctatgtcacgagcaattaagcgactcagaggaagaaaacgatgaaatagatggagttaatcatcaacatttaccagcccgacgagccgaaccacaacgtcacacaatgttgtgtatgtgttgtaagtgtgaagccagaattgagctagtagtagaaagctcagcagacgaccttcgagcattccagcagctgtttctgagcaccctgtcctttgtgtgtccgtggtgtgcatcccagcagtaa

SEQ ID NO: 4 - protein, 105 aa, Human papillomavirus type 18
  REGION 1..105
MYGPKATLQDIVLHLEPQNEIPVDLLCHEQLSDSEEENDEIDGVNHQHLPARRAEPQRHTMLCMCCKCEARIELVVESSADDLRAFQQLFLSTLSFVCPWCASQQ

SEQ ID NO: 5 - DNA, 481 bp, Human papillomavirus type 31
  misc_feature 1..481
  source 1..481: mol_type = other DNA
atgcgtggagaaacacctacgttgcaagactatgtgttagatttgcaacctgaggcaactgacctctactgttatgagcaattacccgacagctcagatgaggaggatgttatagacagtccagctggacaagcaaaaccggacacatccaattacaatatcgttaccttttgttgtcagtgtgagtctacacttcgtttgtgtgtacagagcacacaagtagatattcgcatattgcaagagctgttaatgggctcatttggaatcgtgtgccccaactgttctactagactgtaactacaatggctgatccagcaggtacagatggggaggggacgggatgcaatggttggttttatgtagaagcagtaattgacagacagacaggggacaacatttcagaggacgaaaatgaagacagtagtgatactggggaggatatggttgactttattgacaattgtaatgtatacaacaatca

SEQ ID NO: 6 - protein, 98 aa, Human papillomavirus type 31
  REGION 1..98
MRGETPTLQDYVLDLQPEATDLYCYEQLPDSSDEEDVIDSPAGQAKPDTSNYNIVTFCCQCESTLRLCVQSTQVDIRILQELLMGSFGIVCPNCSTRL

SEQ ID NO: 7 - DNA, 321 bp, Human papillomavirus type 45
  misc_feature 1..321
  source 1..321: mol_type = other DNA
atgcatggaccccgggcaacactgcaagaaattgtattgcatttggaacctcagaatgaattagatcctgttgacctgttgtgttacgagcaattaagcgagtcagaggaggaaaacgatgaagcagatggagttagtcatgcacaactaccagcccgacgagccgaaccacagcgtcacaaaattttgtgtgtatgttgtaagtgtgacggcagaattgagcttacagtagagagctcggcagatgaccttagaacactacagcagctgtttttgagcaccttgtcctttgtgtgtccgtggtgtgcaactaaccaataa

SEQ ID NO: 8 - protein, 106 aa, Human papillomavirus type 45
  REGION 1..106
MHGPRATLQEIVLHLEPQNELDPVDLLCYEQLSESEEENDEADGVSHAQLPARRAEPQRHKILCVCCKCDGRIELTVESSADDLRTLQQLFLSTLSFVCPWCATNQ

SEQ ID NO: 9 - DNA, 1299 bp, Homo sapiens
  source 1..1299: mol_type = other DNA
atgaccacctccatccgccagttcacctcctccagctccatcaagggctcctccggcctggggggcggctcgtcccgcacctcctgccggctgtctggcggcctgggtgccggctcctgcaggctgggatctgctggcggcctgggcagcaccctcgggggtagcagctactccagctgctacagctttggctctggtggtggctatggcagcagctttgggggtgttgatgggctgctggctggaggtgagaaggccaccatgcagaacctcaatgaccgcctggcctcctacctggacaaggtgcgtgccctggaggaggccaacactgagctggaggtgaagatccgtgactggtaccagaggcaggccccggggcccgcccgtgactacagccagtactacaggacaattgaggagctgcagaacaagatcctcacagccaccgtggacaatgccaacatcctgctacagattgacaatgcccgtctggctgctgatgacttccgcaccaagtttgagacagagcaggccctgcgcctgagtgtggaggccgacatcaatggcctgcgcagggtgctggatgagctgaccctggccagagccgacctggagatgcagattgagaacctcaaggaggagctggcctacctgaagaagaaccacgaggaggagatgaacgccctgcgaggccaggtgggtggtgagatcaatgtggagatggacgctgccccaggcgtggacctgagccgcatcctcaacgagatgcgtgaccagtatgagaagatggcagagaagaaccgcaaggatgccgaggattggttcttcagcaagacagaggaactgaaccgcgaggtggccaccaacagtgagctggtgcagagtggcaagagtgagatctcggagctccggcgcaccatgcaggccttggagatagagctgcagtcccagctcagcatgaaagcatccctggagggcaacctggcggagacagagaaccgctactgcgtgcagctgtcccagatccaggggctgattggcagcgtggaggagcagctggcccagcttcgctgcgagatggagcagcagaaccaggaatacaaaatcctgctggatgtgaagacgcggctggagcaggagattgccacctaccgccgcctgctggagggagaggatgcccacctgactcagtacaagaaagaaccggtgaccacccgtcaggtgcgtaccattgtggaagaggtccaggatggcaaggtcatctcctcccgcgagcaggtccaccagaccacccgctga

SEQ ID NO: 10 - protein, 432 aa, Homo sapiens
MTTSIRQFTSSSSIKGSSGLGGGSSRTSCRLSGGLGAGSCRLGSAGGLGSTLGGSSYSSCYSFGSGGGYGSSFGGVDGLLAGGEKATMQNLNDRLASYLDKVRALEEANTELEVKIRDWYQRQAPGPARDYSQYYRTIEELQNKILTATVDNANILLQIDNARLAADDFRTKFETEQALRLSVEADINGLRRVLDELTLARADLEMQIENLKEELAYLKKNHEEEMNALRGQVGGEINVEMDAAPGVDLSRILNEMRDQYEKMAEKNRKDAEDWFFSKTEELNREVATNSELVQSGKSEISELRRTMQALEIELQSQLSMKASLEGNLAETENRYCVQLSQIQGLIGSVEEQLAQLRCEMEQQNQEYKILLDVKTRLEQEIATYRRLLEGEDAHLTQYKKEPVTTRQVRTIVEEVQDGKVISSREQVHQTTR

SEQ ID NO: 11 - DNA, 493 bp, Homo sapiens
  source 1..493: mol_type = other DNA
gcggcgcgccattaaccgccagatttgaatcgcgggacccgttggcagaggtggcggcggcggcatgggtgccccgacgttgccccctgcctggcagccctttctcaaggaccaccgcatctctacattcaagaactggcccttcttggagggctgcgcctgcaccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcggagaaagtgcgccgtgccatcgagcagctggctgccatggattga

SEQ ID NO: 12 - protein, 142 aa, Homo sapiens
MGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIAKETNNKKKEFEETAEKVRRAIEQLAAMD

SEQ ID NO: 13 - DNA, 1518 bp, Human papillomavirus type 16
  misc_feature 1..1518
  source 1..1518: mol_type = other DNA
atgtctctttggctgcctagtgaggccactgtctacttgcctcctgtcccagtatctaaggttgtaagcacggatgaatatgttgcacgcacaaacatatattatcatgcaggaacatccagactacttgcagttggacatccctattttcctattaaaaaacctaacaataacaaaatattagttcctaaagtatcaggattacaatacagggtatttagaatacatttacctgaccccaataagtttggttttcctgacacctcattttataatccagatacacagcggctggtttgggcctgtgtaggtgttgaggtaggtcgtggtcagccattaggtgtgggcattagtggccatcctttattaaataaattggatgacacagaaaatgctagtgcttatgcagcaaatgcaggtgtggataatagagaatgtatatctatggattacaaacaaacacaattgtgtttaattggttgcaaaccacctataggggaacactggggcaaaggatccccatgtaccaatgttgcagtaaatccaggtgattgtccaccattagagttaataaacacagttattcaggatggtgatatggttgatactggctttggtgctatggactttactacattacaggctaacaaaagtgaagttccactggatatttgtacatctatttgcaaatatccagattatattaaaatggtgtcagaaccatatggcgacagcttatttttttatttacgaagggaacaaatgtttgttagacatttatttaatagggctggtactgttggtgaaaatgtaccagacgatttatacattaaaggctctgggtctactgcaaatttagccagttcaaattattttcctacacctagtggttctatggttacctctgatgcccaaatattcaataaaccttattggttacaacgagcacagggccacaataatggcatttgttggggtaaccaactatttgttactgttgttgatactacacgcagtacaaatatgtcattatgtgctgccatatctacttcagaaactacatataaaaatactaactttaaggagtacctacgacatggggaggaatatgatttacagtttatttttcaactgtgcaaaataaccttaactgcagacgttatgacatacatacattctatgaattccactattttggaggactggaattttggtctacaacctcccccaggaggcacactagaagatacttataggtttgtaacatcccaggcaattgcttgtcaaaaacatacacctccagcacctaaagaagatccccttaaaaaatacactttttgggaagtaaatttaaaggaaaagttttctgcagacctagatcagtttcctttaggacgcaaatttttactacaagcaggattgaaggccaaaccaaaatttacattaggaaaacgaaaagctacacccaccacctcatctacctctacaactgctaaacgcaaaaaacgtaagctgtaa

SEQ ID NO: 14 - protein, 505 aa, Human papillomavirus type 16
  REGION 1..505
MSLWLPSEATVYLPPVPVSKVVSTDEYVARTNIYYHAGTSRLLAVGHPYFPIKKPNNNKILVPKVSGLQYRVFRIHLPDPNKFGFPDTSFYNPDTQRLVWACVGVEVGRGQPLGVGISGHPLLNKLDDTENASAYAANAGVDNRECISMDYKQTQLCLIGCKPPIGEHWGKGSPCTNVAVNPGDCPPLELINTVIQDGDMVDTGFGAMDFTTLQANKSEVPLDICTSICKYPDYIKMVSEPYGDSLFFYLRREQMFVRHLFNRAGTVGENVPDDLYIKGSGSTANLASSNYFPTPSGSMVTSDAQIFNKPYWLQRAQGHNNGICWGNQLFVTVVDTTRSTNMSLCAAISTSETTYKNTNFKEYLRHGEEYDLQFIFQLCKITLTADVMTYIHSMNSTILEDWNFGLQPPPGGTLEDTYRFVTSQAIACQKHTPPAPKEDPLKKYTFWEVNLKEKFSADLDQFPLGRKFLLQAGLKAKPKFTLGKRKATPTTSSTSTTAKRKKRKL